(19) United States
(12) Patent Application Publication
(10) Pub. No.: US 2022/0119759 A1
(43) Pub. Date: Apr. 21, 2022

(54) Cascade Tangential Flow Filtration Systems for Perfusion of Cell Culture

(71) Applicant: EMD Millipore Corporation, Burlington, MA (US)

(72) Inventors: Ying Li, Needham, MA (US); Alison Dupont, Lyndeborough, MA (US); Amy Wood, Concord, MA (US); Allyson Fournier, West Newbury, MA (US); Akshat Gupta, Tewksbury, MA (US); Hiral Gami, Westford, MA (US)

(21) Appl. No.: 17/073,465

(22) Filed: Oct. 19, 2020

(51) Int. Cl. C12M 1/00

(52) U.S. Cl. CPC C12M 47/10; C12M 29/04; C12M 29/18; C12M 29/10

(57) ABSTRACT

A multi-stage TFF apparatus, comprising a bioreactor capable of holding cell culture media; a first stage TFF device in fluid communication with the bioreactor; a storage tank in fluid communication with the first stage TFF device; a first recirculating loop in fluid communication with the first stage TFF device and the bioreactor; a second stage TFF device, in fluid communication with and downstream from the storage tank; and a second recirculating loop in fluid communication with the second stage TFF device and the storage tank.

### BACKGROUND

### Field of the Disclosure

Embodiments of the present disclosure relate to the processing of biological fluids. More particularly, embodiments disclosed relate to multi-stage tangential flow systems and processes for the culturing, filtration and perfusion of biological fluids.

### Description of the Related Art

Methods and strategies for growing, feeding, and maintaining cultures, such as monoclonal antibodies, cells, viruses, and other biological products that have positive impacts on cell viability, are varied and difficult to develop. In particular, any change or loss of control in upstream processes can affect the downstream processes, such as concentration and collection of cells from a biological fluid or culture. A typical cell culture experiences exponential growth, wherein cell density is increased. Subsequently, exponential cell growth slows and product titer increases. In batch processes, a cell culture is maintained for set periods of time, followed by harvesting the entire culture within a batch. In continuous harvest systems, e.g., perfusion processes, where spent culture is removed and replaced by fresh media, cell culture permeate, i.e., product, is collected from the cell culture on a continuous basis throughout the process. Perfusion culture can achieve higher cell density, run for longer duration, and improve product quality, which makes it a key enabler of upstream process intensification. On the other hand, higher amounts of waste are also generated from perfusion cell culture process and pose challenges in downstream purification. Upstream and downstream processes are, therefore, subject to balancing the interests of processing times, product concentrations, and quality.

There is a lack of effective methods to address these problems. For example, alternating tangential flow (ATF) systems comprising a small pore size microfiltration membrane as a cell retention device results in significant retention of high molecular weight products within the bioreactor over long term operation. Because of this, the filters must be replaced multiple times during a perfusion cell culture process, increasing costs, labor requirements, and risks of contamination. Moreover, prolonged retention of products, as is necessary, in the bioreactor may have a negative impact on product quality. Further still, limitation of ATF systems includes lack of scalability. Such systems are undersized for large scale manufacturing processes, inevitably impacting the performance.

Another prior art invention consists of tangential flow depth filtration devices. These devices suffer from lessened quality of product. Specifically, a perfusate from open pore membrane filters, i.e., tangential flow depth filters or hollow fiber filters having an average pore size greater than 5 μm, typically have unacceptable turbidity properties, ranging from 10 to 80 NTU, and require additional clarification before feeding into the next downstream operation, such as Protein A chromatography, to avoid column clogging and improve resin capacity. The capacity of conventional depth filter (operating in normal flow filtration mode) is low, i.e., less than 1000 L/m² and, therefore, a large number of filters are required to clarify the perfusate. In addition, conventional depth filters cannot be connected inline to a perfusion bioreactor in a sterilized and closed environment. Subsequently, replacement and flushing of filters on an at least daily basis is required.

Single-pass tangential flow filtration systems can achieve higher permeate conversion through a single pass therefore avoid recirculation loops. However, these SPTFF systems comprise very long, staged flow paths and employ membranes for the concentration of proteins, making their applications limited. The membranes for SPTFF can be configured with step-wise decreases in cross-sectional membrane areas resulting in higher concentration factors at a given feed flux, compared with constant-area cross-sectional membrane areas but cannot be easily used for cell retention. SPTFF is used to achieve higher conversion of feed into permeate in a single pass (for concentration/diafiltration of products). For the case of perfusion cell culture, the conversion of feed into permeate is very low (typically <3%), therefore, practical use of SPTFF in this application is lacking. Furthermore, it may result in more rapid membrane fouling due to the long flow path and lower feed flux compared with typical TFF devices.

Methods and cell culturing apparatus for producing antibodies, therapeutic proteins, blood factors and enzymes that overcome the deficiencies of past prior art attempts and balance the interests of processing duration, quality and concentration while providing increased product recovery represent an advance in the art. Methods and apparatus that are capable of providing higher product yields and quality, that are linearly scalable across different scales, that provide higher throughput through filters and/or operate in closed, continuous processing represent inventive advances in the art.

### SUMMARY OF SOME EMBODIMENTS

A two-stage tangential flow filtration (TFF) system and method for using same in perfusion cell culture processes; substantially as shown in and/or described in connection with at least one of the figures, as set forth more completely in the claims, are disclosed. In some embodiments, a first TFF device retains viable cells within a bioreactor while continuously removing spent media and products from the bioreactor using a large pore size membrane, e.g., approximately 5-10 μm. A permeate from the first TFF device is fed to a second TFF device, wherein the second TFF device clarifies the permeate using a small pore size microfiltration membrane, e.g., <0.2 μm. In some embodiments, the first TFF device and the second TFF device operate within a continuous, and/or closed, sterilized system. In some embodiments, a cascading TFF system for cell retention and clarification in perfusion cell culture processing is disclosed. In some embodiments, a permeate from a cascade TFF system has been clarified by microfiltration (<0.2 μm) and directly fed into a next downstream unit operation. In some embodiments, the cascade TFF system is scalable. In some embodiments, a first stage TFF device allows complete or nearly complete product passage for long duration perfusion cell culture processing and a second stage TFF device provides clarification of a perfusate containing high concentrations of cellular debris, while maintaining high product recovery. In some embodiments, a two-stage tangential flow filtration system that enables the use of perfusion to produce biological products, such as antibodies, therapeutic proteins, blood factors and enzymes, and the like, and generates a product stream that can be delivered to downstream processing without further clarification.

Various novel and inventive features of the present disclosure, as well as details of exemplary embodiments thereof, will be more fully understood from the following description and drawings.

### BRIEF DESCRIPTION OF THE FIGURES

FIG. 1 depicts a cell-culturing system comprising a bioreactor, a storage tank, and a multi-stage TFF apparatus according to embodiments of the disclosure;

FIG. 2 depicts a graph showing a cell culture performance, viable cell density and viability as a function of time within a first TFF device, embodiments according to the disclosure;

FIG. 3 depicts a graph of product sieving by percent, according to embodiments of the disclosure;

FIG. 4 depicts a graph of clarification performance of a second TFF device, according to embodiments of the disclosure;

FIG. 5 depicts a graph of turbidity of a product concentration in a permeate and a product concentration of a feed, according to embodiments of the disclosure; and

FIG. 6 depicts a graph of a performance of a prior art 0.2 μm PES membrane ATF device with the same cell culture.

### DETAILED DESCRIPTION OF SOME EMBODIMENTS

So the manner in which the features disclosed herein can be understood in detail, a more particular description of the embodiments of the disclosure, briefly summarized above, may be had by reference to the appended drawings. It is to be noted, however, that the appended drawings illustrate only typical embodiments of this disclosure and are therefore not to be considered limiting of its scope, for the embodiments described and shown may admit to other equally effective embodiments. It is also to be understood that elements and features of one embodiment may be found in other embodiments without further recitation and that identical reference numerals are sometimes used to indicate comparable elements that are common to the figures.

The terms “bioreactor,” “bag,” and “container” are generally used interchangeably within this disclosure. A flexible bioreactor, bag, or container connotes a flexible vessel that can be folded, collapsed, and expanded and/or the like, capable of containing, for example, a biological fluid. A single use bioreactor, bag, or container, typically also flexible, is a vessel that is used once and discarded.

The term “sterile” is defined as a condition of being free from contaminants and, particularly within the bioprocessing industry, free from undesirable viruses, bacteria, germs, and other microorganisms.

The term “upstream” is defined as first step processes in the processing of biological materials, such as microbes/cells, mAbs, proteins, including therapeutic proteins, viral vectors, etc., are grown or inoculated in bioreactors within cell culture media, under controlled conditions, to manufacture certain types of biological products.

The term “downstream” indicates those processes in which biological products are harvested, purified, formulated and packaged.

The term “clarification” is defined as a downstream process, wherein large insoluble contaminants, usually whole cells and cell debris are separated from the feedstock or harvest.

The term “purification” is defined as a downstream process, wherein bulk contaminants and impurities, including host cell proteins, DNA and process residuals are removed from the product stream.

The term “polishing” is defined as a downstream process, wherein trace contaminants or impurities that resemble the product closely in physical and chemical properties are eliminated from the purified product stream.

The term “chromatography” is defined as a downstream process suitable for biological chromatographic techniques, comprising, but not limited to, protein A chromatography, affinity chromatography, hydrophobic interaction chromatography, column chromatography, and ion exchange chromatography, e.g., anion exchange chromatography, and cation exchange chromatography.

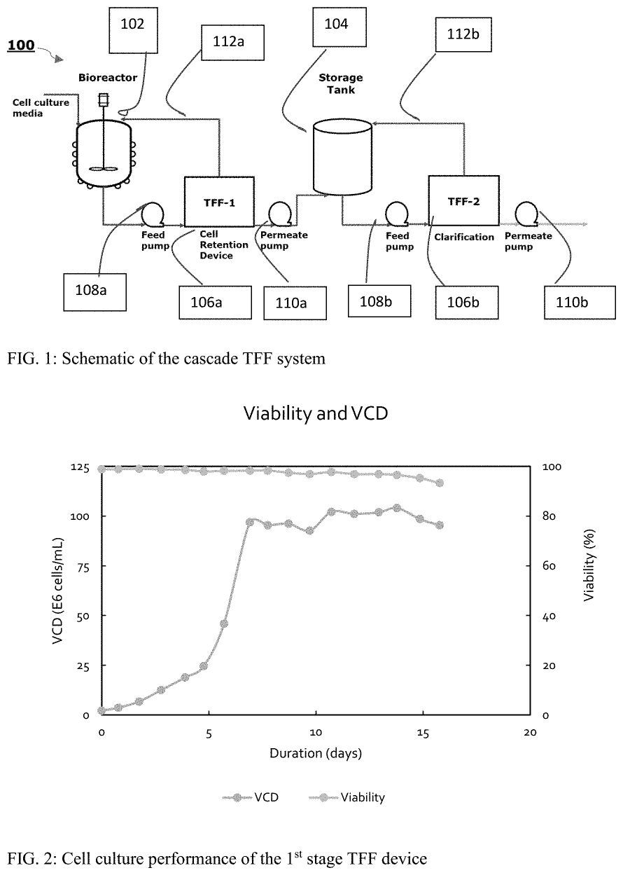

FIG. 1 depicts a cell-culturing system comprising a bioreactor, a storage tank, and a multi-stage TFF apparatus; according to embodiments of the disclosure. FIG. 1 depicts a cell-culturing system 100 comprising a bioreactor 102, a storage tank 104. and, collectively, a multi-stage TFF apparatus comprising a first stage TFF device 106a, and a second stage TFF device 106b; according to embodiments of the disclosure. The bioreactor 102 is capable of culturing cells that produce antibodies, viruses, and/or other biologics. The bioreactor 102 is capable of being operated under perfusion conditions. For example, cell culture media is supplied periodically or continuously as cell cultures to the first stage TFF device 106a. In some embodiments, the downstream processing is a first stage TFF device 106a. In some embodiments, the first stage TFF device 106a is a cell retention device externally connected to the bioreactor to retain the viable cells within the bioreactor while continuously removing the spent media and product.

The cell-culturing system 100 further comprises a storage tank 104. The storage tank 104 is disposed in fluid communication and between the first stage TFF device 106a, which is, for example, a cell retention device and a second stage TFF device 106b. A first feed pump 108a is capable of delivering, for e.g., a product stream directly to downstream processing apparatus without further clarifying the product stream. A permeate is delivered from the first stage TFF device 106a to the storage tank 104 via a first permeate pump 110a. The first stage TFF device 106a also returns fluid to the bioreactor 102 via a first recirculating loop 112a. A feed from the storage tank 104 is delivered, via a feed pump 108b, to the second stage TFF device 106b for clarification. A loop returns some fluid to the storage tank 104 using a second permeate pump 108b and a second recirculating loop 112b.

According to embodiments of the disclosure, the first stage TFF 106a device is a cell retention device externally connected to the bioreactor capable of retaining the viable cells within the bioreactor while removing, optionally continuously, the spent media and product. A large pore size membrane (5-10 μm) is used to mitigate membrane fouling and prevent loss of product sieving over a long period of time during perfusion cell culture. The permeate from the first stage TFF 106a is stored in an intermediate storage tank, from which the feed is introduced continuously to the second stage TFF 106b, and flow rate in and out each TFF stage is similar to maintain the mass balance. The second stage TFF device 106b separates cell debris and other impurities from the product molecule using a small pore size microfiltration membrane (for example, a ≤0.2 μm). The processed product can be fed into the next unit operation (for e.g., Protein A chromatography for monoclonal antibodies (mAbs) and/or other downstream biological processes known to those in the art) without further clarification.

2 depicts a graph showing a cell culture performance, viable cell density and viability as a function of time within a first TFF device, embodiments according to the disclosure. As can be seen, the viability of the cells remains nearly constant at approximately 95% during a 15-day duration. Also, a viable cell density reaches 100×10⁶ cells/mL after 6 days and maintains this level, or increases, over a subsequent 9 days.

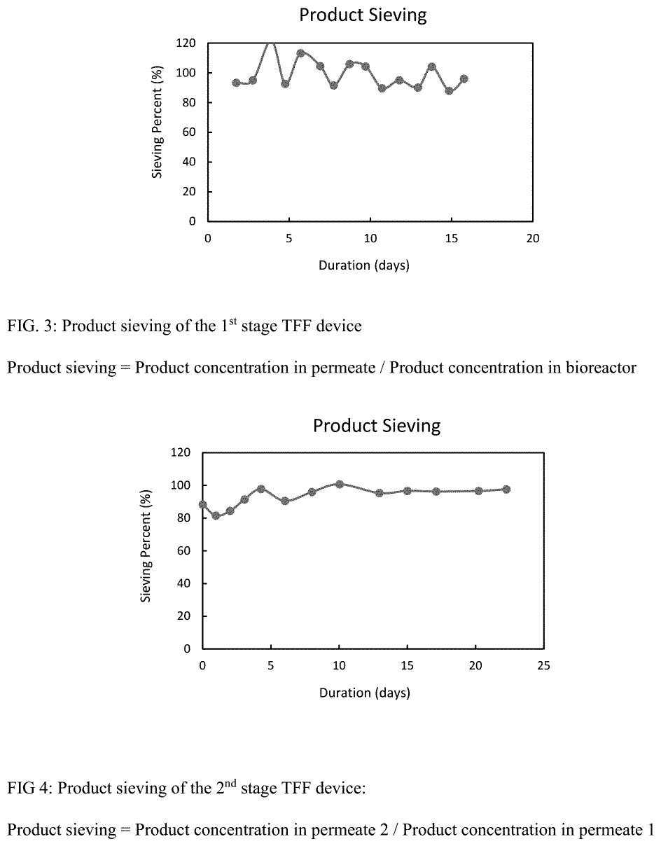

FIG. 3 depicts a graph of product sieving by percent, according to embodiments of the disclosure. As can be seen, a product sieving percent % (defined as product concentration in the first permeate divided by the product concentration in the bioreactor), maintains at around 100% over 15 days, which indicates that nearly all products passed through the membrane during the entire cell culture process.

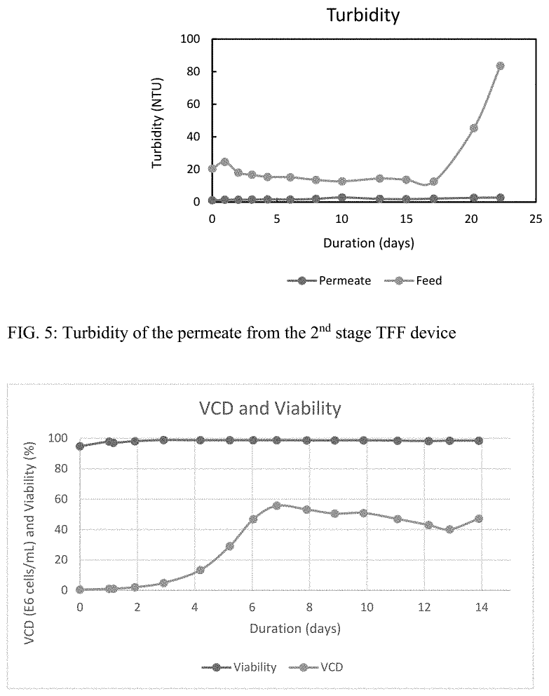

FIG. 4 depicts a graph of clarification performance of a second TFF device, according to embodiments of the disclosure. As can be seen, the product sieving of a clarification process (defined as product concentration in the second permeate divided by the product concentration in the first permeate) by the second stage TFF device products approximately 85-100% sieving percent from time t=0 and remains nearly constant over 22 days. The overall product sieving across the two-stage TFF is greater than 85% throughout the entire process. In comparison, when operating at similar cell culture conditions (viable cell density and viability), the product sieving of ATF or TFF system using small pore size microfiltration membrane rapidly drops to below 50% within 7 days of perfusion cell culture (See references 1 and 2, below). This demonstrates that the proposed system successfully addresses the challenge of product retention using existing ATF and TFF systems.

FIG. 5 depicts a graph of turbidity of a product concentration in a permeate and a product concentration of a feed, according to embodiments of the disclosure. As can be seen, a permeate from the second stage TFF device has a turbidity of less than 2 (NUT) from time t=0 over the course of 22 days. This demonstrates that the system is able to operate for more than 3 weeks without turbidity breakthrough and the need to switch filters. Both TFF devices can be pre-sterilized and pre-assembled and the whole system can potentially operate in a fully closed, sterilized environment, minimizing the risk of contamination. The permeate from the cascade TFF system as described herein can load directly onto next purification unit operation(s) without further clarification. In comparison, the turbidity of perfusates from open pore membrane filters (i.e., tangential flow depth filters or hollow fiber filters having an average pore size greater than 5 μm) typically range from 10 to 80 NTU, resulting in the need for an additional depth filtration step to remove particles and cell debris from the perfusion permeate stream (See references 2 and 3, below).

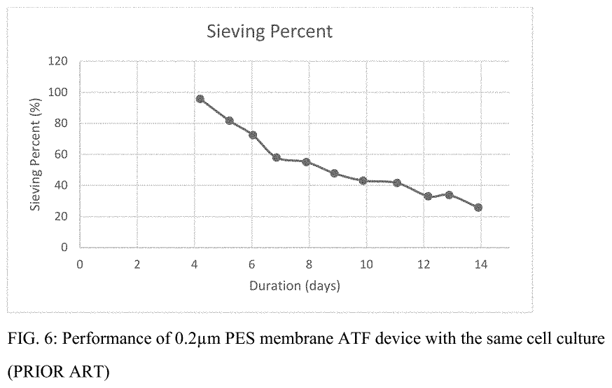

FIG. 6 depicts a graph of a performance of a 0.2 μm PES membrane prior art ATF device with the same cell culture. As can be seen, the cascade TFF system and methods described herein outperform prior art ATF devices and methods with respect to sieving.

Embodiments of the disclosure also comprise methods for perfusing cells within a cell culture media. For example, some embodiments of the disclosure include a method for processing biological fluids, comprising providing biological fluids within a cell culture media to a bioreactor and growing biological cells therein; delivering the biological fluid to a first stage TFF device, wherein cells are retained within the first stage TFF device and producing a permeate; perfusing the biological fluid by returning the biological fluid to the bioreactor via first recirculating loop; delivering the permeate to a storage tank; feeding the permeate from the storage tank to a second stage TFF device, wherein the permeate is clarified, wherein some of the permeate is recirculated via a second recirculating loop to the storage tank and a second permeate is delivered to final fill container for storage as a biological product.

Reference throughout this specification to “one embodiment,” “certain embodiments,” “one or more embodiments,” “some embodiments,” or “an embodiment” indicates that a feature, structure, material, or characteristic described in connection with the embodiment is included in at least one embodiment of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Although some embodiments have been discussed above, other implementations and applications are also within the scope of the following claims. Although the specification describes, with reference to particular embodiments, it is to be understood that these embodiments are merely illustrative of the principles and applications of the present disclosure. It is therefore to be further understood that numerous modifications may be made to the illustrative embodiments and that other arrangements and patterns may be devised without departing from the spirit and scope of the embodiments according to the disclosure. Furthermore, particular features, structures, materials, or characteristics may be combined in any suitable manner in any one or more of the embodiments.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

Reference (1): Wang, S. B., Godfrey, S., Radoniqi, F., Lin, H., & Coffman, J. (2019). Larger pore size hollow fiber membranes as a solution to the product retention issue in filtration-based perfusion bioreactors. Biotechnology Journal, 14(2), 1800137, which is incorporated by reference in its entirety.

Reference (2): Pinto, N. D., Napoli, W. N., & Brower, M. (2020). Impact of micro and macroporous TFF membranes on product sieving and chromatography loading for perfusion cell culture. Biotechnology and Bioengineering, 117(1), 117-124, which is incorporated by reference in its entirety.

Reference (3): WO 2017/180814 A1 to Coffman, which is incorporated by reference in its entirety.

What is claimed is:

1. A multi-stage TFF apparatus, comprising:
a bioreactor capable of holding cell culture media;
a first stage TFF device in fluid communication with the bioreactor;
a storage tank in fluid communication with the first stage TFF device;
a first recirculating loop in fluid communication with the first stage TFF device and the bioreactor;
a second stage TFF device, in fluid communication with and downstream from the storage tank; and
a second recirculating loop in fluid communication with the second stage TFF device and the storage tank.

2. The multi-stage TFF apparatus of claim 1, further comprising a first feed pump disposed between the bioreactor and the first stage TFF device.

3. The multi-stage TFF apparatus of claim 1, further comprising a second feed pump disposed between the storage tank and the second stage TFF device.

4. The multi-stage TFF apparatus of claim 1, further comprising a first permeate pump disposed between the first stage TFF device and the storage tank.

5. The multi-stage TFF apparatus of claim 1, further comprising a second permeate pump disposed downstream of the second stage TFF device.

6. The multi-stage TFF apparatus of claim 1, wherein the first stage TFF device is a cell retention device.

7. A method for processing biological fluids, comprising:
providing biological fluids within a cell culture media to a bioreactor and growing biological cells therein;
delivering the biological fluid to a first stage TFF device, wherein cells are retained within the first stage TFF device and producing a permeate;
perfusing the biological fluid by returning the biological fluid to the bioreactor via first recirculating loop;
delivering the permeate to a storage tank;
feeding the permeate from the storage tank to a second stage TFF device, wherein the permeate is clarified, wherein some of the permeate is recirculated via a second recirculating loop to the storage tank and a second permeate is delivered to final fill container for storage as a biological product.
